(19) United States
(12) Patent Application Publication
(10) Pub. No.: US 2015/0342519 A1
(43) Pub. Date: Dec. 3, 2015

(54) SYSTEM AND METHOD FOR DIAGNOSING MEDICAL CONDITION

(71) Applicant: Huneo, LLC, Fremont, CA (US)

(72) Inventor: Alex Zhiqiang Zheng, Fremont, CA (US)

(21) Appl. No.: 14/722,627

(22) Filed: May 27, 2015

Related U.S. Application Data
(60) Provisional application No. 62/004,125, filed on May 28, 2014.

(51) Int. Cl. A61B 5/00; A61B 5/11; A61B 7/00

(52) U.S. Cl. CPC A61B 5/4818; A61B 7/003; A61B 5/4809; A61B 5/1112; A61B 5/6898; A61B 2560/0242

(57) ABSTRACT

A system and method to diagnose a medical condition using sound recordings. Included is a method to diagnose sleep apnea by computing a sleep index based on an analysis of snoring of the patient. This can be accomplished via a recording of the patient sleep sounds using sensors (e.g., microphones) available in a smart mobile device. A sleep index is calculated that is highly correlated to the Apnea-Hypopnea Index and can be used as an objective diagnosis indicator for sleep apnea.

### CROSS-REFERENCES TO RELATED APPLICATIONS

This application claims the benefit of provisional patent application 62/004,125 filed on May 28, 2014 and incorporated herein by reference.

The disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

Sleep apnea is a type of sleep disorder characterized by pauses in breathing or instances of reduced breathing during sleep. Each pause in breathing, called an apnea, can last from at least ten seconds to several minutes, and may occur 5 to 30 times or more an hour. Similarly, each reduced breathing event, when accompanied by a corresponding reduction in blood oxygen saturation (minimum of 3 or 4% depending on the scoring guidelines), is called a hypopnea.

The diagnosis of sleep apnea is currently based on the results of a formal sleep study (polysomnography or reduced channels home based test). It aims at establishing an objective diagnosis indicator linked to the quantity of apneic events per hour of sleep. Apnea Hypopnea Index (AHI) is such a diagnosis indicator, which equals the total number of apnea and hypopnea events divided by the number of hours of sleep. AHI values are typically categorized as 5-15 per hour is diagnosed as mild; 15-30 per hour is diagnosed as moderate; and greater than 30 per hour is diagnosed as severe. Respiratory Disturbance Index (RDI) is another diagnosis indicator. RDI equals the total number of apnea, hypopnea, and respiratory-effort related arousal events divided by the total number of hours of sleep.

It has been estimated that 3-15% of adult population in the US has sleep apnea. Due to high expense of sleep studies and intrusiveness of the diagnostic equipment, many of them are not diagnosed. There is a significant need for a simpler and cheaper method to screen and diagnose sleep apnea.

### SUMMARY

Provided is a method to compute a sleep index based on a measurement of snoring of the patient. This can be done using a recording of the patient sleep sounds using sensors (e.g., microphones) available in today's smart mobile devices. A sleep index is calculated that is highly correlated to the Apnea-Hypopnea Index and can therefore be used as an objective diagnosis indicator for sleep apnea.

Provided are a plurality of example embodiments, including, but not limited to, a system for diagnosing a medical condition of a patient, said system comprising: a computing device comprising first specialized software and a microphone, said computing device being configured for executing said specialized software such that the computing device collects and records sounds of the patient and the patient's surroundings while the patient is sleeping, where the computing device is configured to connect to a communication network.

The system also comprises an analysis device comprising second specialized software for receiving the recorded sounds collected by the computing device using the communication network, wherein said analysis device executes the second specialized software to analyze the received sounds for diagnosing a condition of sleep apnea in the patient.

Also provided is a system for diagnosing a medical condition of a patient, said system comprising: a computing device comprising first specialized software and a microphone, said computing device being configured for executing said specialized software such that the computing device collects and records snoring sounds of the patient while the patient is sleeping. The system is configured for executing second specialized software to analyze the snoring sounds for diagnosing a condition of sleep apnea in the patient.

Still further provided is a system for diagnosing a medical condition of a patient, said system comprising: a personal computing device comprising first specialized software, a microphone, and an additional sensor, said computing device being configured for executing said specialized software such that the personal computing device uses the microphone to collect and record snoring sounds of the patient while the patient is sleeping, and wherein said personal computing device is also configured for executing said specialized software such that the personal computing device uses the additional sensor to collect and record additional data while the patient is sleeping. The computing device is configured to connect to a communication network.

The system can also comprise an analysis device remotely located from said personal computing device, said analysis device comprising second specialized software for receiving the recorded snoring sounds and additional data collected by the computing device using the communication network, wherein said analysis device executes the second specialized software to analyze the snoring sounds and additional data for diagnosing a condition of sleep apnea in the patient.

Further provided is a method of diagnosing a medical condition of a patient, said system comprising the steps of:
collecting and recording snoring sounds of a sleeping patient;
analyzing the recorded snoring sounds of the sleeping patient to detect patterns in the snoring sounds; and
diagnosing a condition of sleep apnea in the patient by analyzing the patterns in the snoring sounds.

Still further provided is the above method wherein additional sounds are collected and recorded from which the snoring sounds are extracted.

Also provided are additional example embodiments, some, but not all of which, are described hereinbelow in more detail.

### BRIEF DESCRIPTION OF THE DRAWINGS

The features and advantages of the example embodiments described herein will become apparent to those skilled in the art to which this disclosure relates upon reading the following description, with reference to the accompanying drawings, in which:

FIG. 1 is a diagram showing an example system for performing the example diagnostic method;

FIG. 2 is a block diagram showing an example smart device for collecting the data for analysis for supporting the diagnostic method; and

FIG. 3 show example data patterns that can be used by the diagnostic method for the example diagnosis.

### DETAILED DESCRIPTION OF THE EXAMPLE EMBODIMENTS

Provided is an example system for performing a diagnostic procedure for detecting the presence of sleep apnea in a patient.

As will be appreciated by one of skill in the art, the example embodiments may be actualized as, or may generally utilize, a method, system, computer program product, or a combination of the foregoing. Accordingly, any of the embodiments may take the form of an entirely hardware embodiment, an entirely software embodiment (including firmware, resident software, microcode, etc.) for execution on hardware, or an embodiment combining software and hardware aspects that may generally be referred to as a “system.” Generally, the “system” will comprise a remotely located server with storage capability such as one or more databases that interact with a plurality of remote devices via a communication network such as the Internet, an intranet, or another communication network such as a cellular network. The smart devices that will be used in the location of the patient include any of a plurality of computing devices, such as smart phones, phablets, tablets, or personal computers, for example. The remote devices will execute software (one or more “apps”) that has been downloaded from the server to each of the remote devices to perform the functions described herein.

Furthermore, some of the embodiments may take the form of a computer program product on a computer-usable storage medium having computer-usable program code embodied in the medium configured for installation on a computing device for execution.

Today's smart devices (e.g., smart phones, watches, PDAs, tablets, phablets, personal computers (PCs), and even smart wearable devices) are widely available in the US and are very multi-functional, being capable of specialized programming for novel uses and applications. Most of these devices, in particular mobile devices such as smart phones, tablets, phablets or PDAs have a number of built-in sensors including: one or more microphones, a light sensor, accelerometer, and a gyroscope. Such sensor can also be used as inputs to a PC. These sensors can be utilized, along with specialized software, to create a new specialized device and method for diagnosing sleep apnea in a convenient and non-intrusive manner.

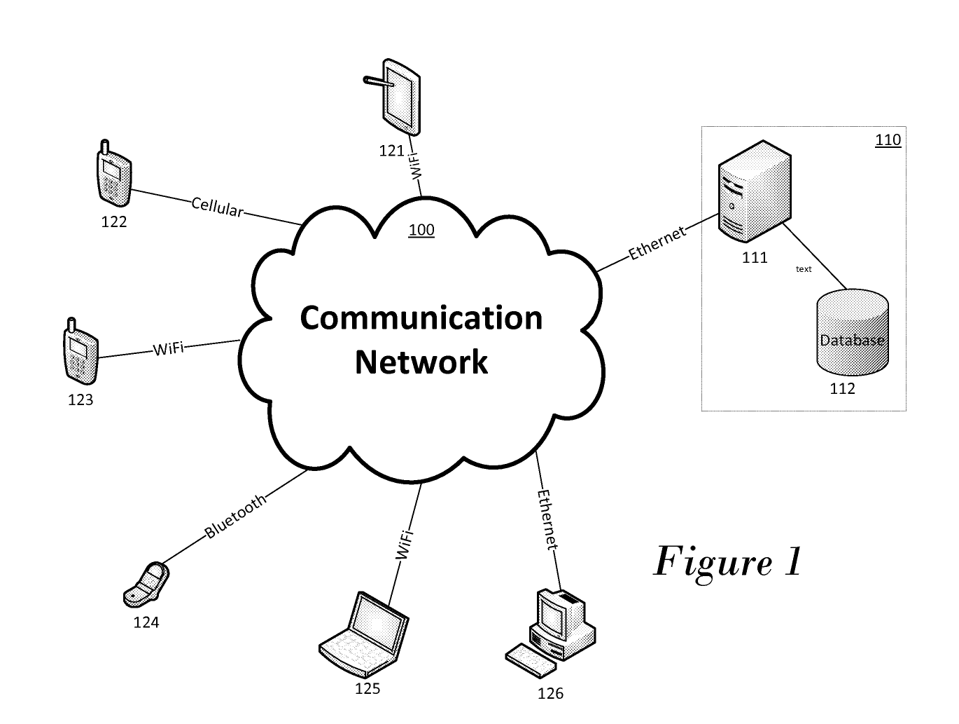

FIG. 1 shows an example system for implemented the disclosed diagnostic process. Various smart computing devices are used for individual patients to collect the sleep data for diagnosing sleep apnea. These smart devices can utilize any computing device capable of executing specialized software for collecting the sleep data, with such devices including such diverse devices as smart phones 122, 123, cell phones 124, tablets 121, laptops 125, PCs 126, among others, and which can connect to a communication network 100 such as the Internet using various communications protocols such as WiFi, cellular networks, Bluetooth, Ethernet, etc. These smart devices then communicate with one or more locally or remotely located analysis devices 110 including an analysis computer 110 and optionally a database 112 for receiving, storing, and performing an analysis on the data received and collected from the smart devices. In particular, smart devices comprised of personal mobile computing devices (e.g., smart phones, phablets, tablets, PDAs, etc.) are particularly useful, considering their ubiquitous nature and configurability with customized applications.

Optionally, the functions of the smart computing device can be combined with the functions of the analysis computer, avoiding the need of an intervening communication network. For example, a smart computing device with sufficient computing capability and storage may be used as a self-contained device to perform all parts of the data collection and diagnostic functions. In particular, this might be done using a computer with external sensors in a clinical environment, for example.

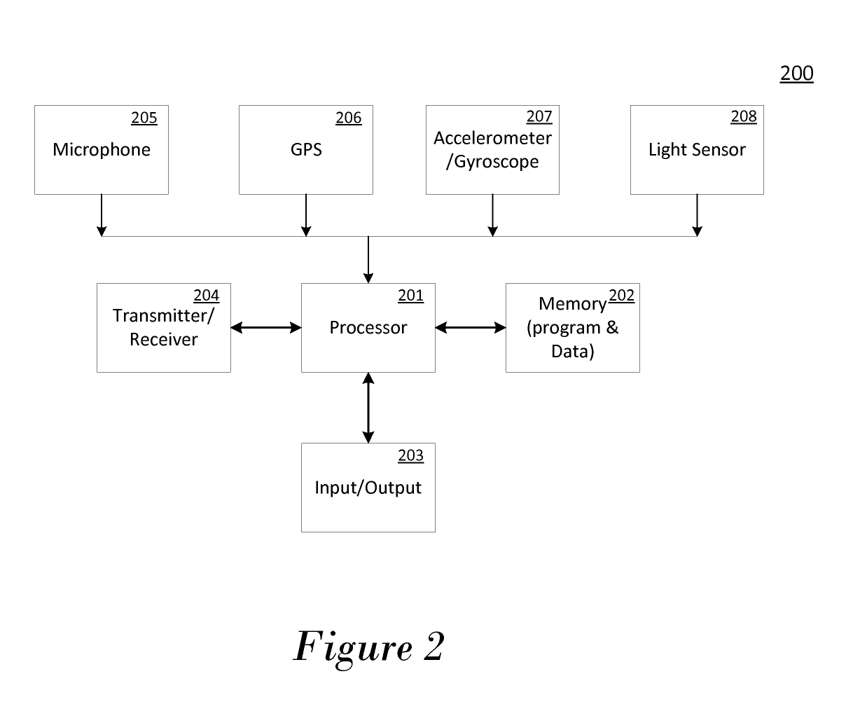

FIG. 2 shows a block diagram of the basic components of an example smart computing device 200, which preferably will be a mobile device. The device will comprise one or more processors 201 and one or more memories 202 for storing the specialized program and data.

The smart device 200 will have a transmitter/receiver 204 for connecting to the communication network to ultimately transmit data to the analysis device 110 (FIG. 1) for performing the medical analysis the collected data.

The smart device 200 also includes one or more input/output interface 203 for communicating with a user, such as a touch screen, keypad, etc.

The smart device 200 also has a plurality of input sensors, such as a microphone 205, GPS subsystem 206, Accelerometer and/or gyroscope 207, and light sensor 208 with which it can collect data from its surroundings.

Together, the smart device 200 with the installed specialized software application installed in memory 202 that can be executed by the processor 201 to configure the device to act as a specialized machine for collecting data from the patient and the patient's surroundings during a monitoring period, in this case while the patient is sleeping.

Any suitable computer usable (computer readable) medium may be utilized for storing the different specialized software applications that are executed by the analysis device 110 and the smart device 200, respectively. The computer usable or computer readable medium may be, for example but not limited to, an electronic, magnetic, optical, electromagnetic, infrared, or semiconductor system, apparatus, device, or propagation medium. More specific examples (a non-exhaustive list) of the computer readable medium would include the following: an electrical connection having one or more wires; a tangible medium such as a portable computer diskette, a hard disk, a random access memory (RAM), a read-only memory (ROM), an erasable programmable read-only memory (EPROM or Flash memory), a compact disc read-only memory (CDROM), cloud storage (remote storage, perhaps as a service), or other tangible optical or magnetic storage device; or transmission media such as those supporting the Internet or an intranet.

The specialized software applications are comprised of computer program code specifically configured for carrying out operations of the example embodiments (i.e., for the smart devices and analysis device(s) of a given system) may be written by conventional means using any computer language, including but not limited to, an interpreted or event driven language such as BASIC, Lisp, VBA, or VBScript, or a GUI embodiment such as visual basic, a compiled programming language such as FORTRAN, COBOL, or Pascal, an object oriented, scripted or unscripted programming language such as Java, JavaScript, Perl, Smalltalk, C++, Object Pascal, C#, Swift, or the like, artificial intelligence languages such as Prolog, a real-time embedded language such as Ada, or even more direct or simplified programming using ladder logic, an Assembler language, or directly programming using an appropriate machine language. Web-based languages such as HTML or any of its many variants may be utilized. Graphical objects may be stored using any graphical storage or compression format, such as bitmap, vector, metafile, scene, animation, multimedia, hypertext and hypermedia, VRML, and other formats could be used. Audio storage could utilize any of many different types of audio and video files, such as WAV, AVI, MPEG, MP3, MP4, WMA, FLAG, MOV, among others. Editing tools for any of these languages and/or formats can be used to create the software.

The computer program instructions of the software and/or scripts comprising the code may be provided to the respective computing device (e.g., a smartphone, tablet, phablet, PC or other device) which includes one or more programmable processors or controllers, or other programmable data processing apparatus, which executes the instructions via the processor of the computer or other programmable data processing apparatus for implementing the functions/acts specified in this document. It should also be noted that, in some alternative implementations, the functions may occur out of the order noted herein. The software applications can be downloaded to the respective devices in a conventional manner, and could be provided by a software vending site such as an Android or Apple app store, for example.

The specialized software application is installed in the smart computing device (in particular a personal computing device that is mobile and portable) for execution that causes the smart device to collect and record sensor data from near or on a patient while the patient is sleeping. The specialized software is executed on the smart computing device to perform a diagnostic method that determines a sleep index that is highly correlated with AHI for diagnosing sleep apnea in the patient. This diagnostic method is based on a recording of the background noises near the patient, including the snoring and other night sounds generated by the patient. Additional data can also be collected, such as light or patient motion information, for example. The diagnostic method is adaptive in terms of the number of sensors available on the smart device.

The smart computing device executes the specialized software application to guide the user (patient) through the following steps to ensure that the recording is of the highest quality possible: (1) Calibrating: (a) the light sensors, (b) movement (e.g., accelerometer) sensors, and (c) and microphone sensors (any other sensors to be used may be similarly calibrated as well).; (2) The user is reminded by the device, such as by a textual or audio message, for example, that only the user (patient) should be sleeping in the room at night during the process or only the user snores when there are multiple people sleeping in the room; (3) The recording/diagnostic application is started.

The user can choose to place the smart computing device or a portion thereof on himself/herself during sleep (when such a device or the portion thereof is properly sized for such use), or anywhere nearby (such as when the device is a larger and/or heavier computing device). In situations where the device (or at least a portion having a sensor for detecting motion/movement) is mounted, worn, or otherwise placed on the user, the device may be better able to determine any movement of the user (patient) during the night, which could prove useful in determining the periods where the sound recording may be most accurate for use in the diagnosis.

During the night, an application that is part of the specialized software executes on the smart device to record the night sounds while the patient is sleeping, along with recording any other data that is detected during this process. The data is correlated with the time of the recording.

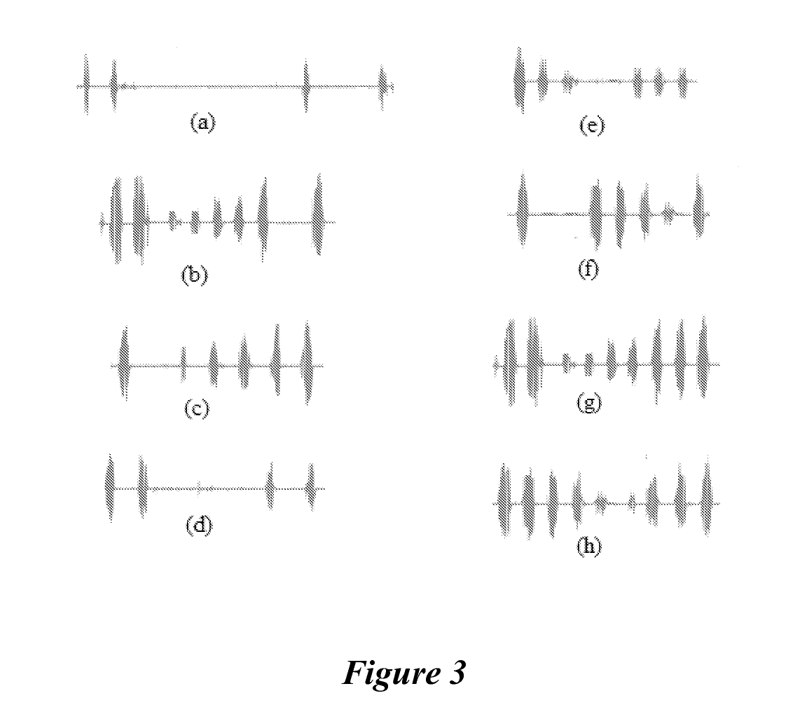

Upon the completion of the data recording process, such as by the end of the night, or after a certain number of hours of recording (which may be predetermined or user selectable), the smart device transmits the recorded data to another analysis device executing additional specialized to act as an analysis engine for storage and analysis of the recorded data. The analysis engine executes an algorithm to determine the sleep index using various rules, such as the example patterns shown in FIG. 3. Optionally, signal processing can be performed on the detected and/or recorded audio data to prevent the reconstruction of the original sounds (e.g., voices) to protect patient privacy but at the same time maintain the desired information to diagnose the medical condition (e.g., sufficient for snore and apnea detection).

Specifically, the analysis to determine the sleep index (i.e., determine whether sleep apnea is occurring) involves: (1) Determining the wake period where the data indicates that there is speaking or other noises/sounds of certain minimum duration indicating that the patient is likely awake; (2) Determining an apnea event where the data indicates that a limited duration of no snore is followed by one or a few snores, which are then followed by another limited period of no snore (see FIG. 3(a)); (3) Determining an apnea event where the data indicates that there is a crescendo of limited duration in snore, followed (as shown in FIG. 3(b)) or preceded (as shown in FIG. 3(c)) by no snore event of a limited duration; (4) Determining an apnea event where the data shows that there is no snore of limited duration (see FIG. 3(d)); (5) Determining an apnea event where the data shows that there is a diminuendo of limited duration in snore, followed (FIG. 3(e)) or preceded (FIG. 3(f)) by no snore of a limited duration; and (6) Determining an apnea event where the data shows that there is a crescendo (FIG. 3(g)) or diminuendo (FIG. 3(h)) in the snore of limited duration.

For each pattern described above, the analysis computes the probability of an apnea event by taking into account the following additional information: (1) The determined snore pattern and characteristics throughout the sleep; (2) a current determined snore amplitude and duration, relative to nearby periods before and after the current snore pattern; and (3) Other sensor recordings—for example, if there is recording for the light sensor and the light is determined to be ON during the period when there is no snore in contrast to determined to be OFF at other times, then the probability of an apnea event is near zero.

After all the patterns are determined and their probabilities computed, an overall sleep index and its confidence interval is computed based on the determined apnea events, which is used to diagnose sleep apnea, if present.

If a recording for data from the gyroscope sensor is available, this data can be used to determine the sleep index for each sleep position as the sensor detects that the person has moved or otherwise changed position. In this way, desirable vs. undesirable sleep positions can also be determined.

As an extension, the smart device can be configured using the specialized software to detect many different (e.g., all) sounds during the process. This can be used to compute a sound level distribution and determine the base background noise. Such a process can be carried out over multiple intervals so that a base background noise level is determined for each interval (for example, the background noise at 1 am differs from 7 am). Then, the base background noise can be subtracted or otherwise extracted from the desired sound data. The desired data sound level can then be scaled based on the calibrated sound level so that the desired sound level is more independent of the specific device and specific patient. Furthermore, the intervals where the sound levels are above some threshold can be determined, which can be used as the sound intervals for determining the snore periods and ultimately for detecting the apneas.

Another extension is to detect and categorize snores and other sounds. The detected sounds can be sorted into three categories based on their characteristics, with example categories being: (1) Snores, determined when their durations are within a certain range (e.g., between 0.1 and 2 seconds) and when they occur at a frequency between 1/6 (one snore per six seconds) and 1/3 (one snore per three seconds) when apnea events are excluded. Also snores may appear in a large number. (2) Talk: gaps between sounds tend to be small; and (3) Sudden sounds (e.g., bed frame squeezing sound from body movement). Usually there is no sound before and after sudden sounds.

Various heuristics can also be deployed to help categorize the sounds (e.g., snores do not follow talk immediately).

Many other example embodiments can be provided through various combinations of the above described features. Although the embodiments described hereinabove use specific examples and alternatives, it will be understood by those skilled in the art that various additional alternatives may be used and equivalents may be substituted for elements and/or steps described herein, without necessarily deviating from the intended scope of the application. Modifications may be necessary to adapt the embodiments to a particular situation or to particular needs without departing from the intended scope of the application. It is intended that the application not be limited to the particular example implementations and example embodiments described herein, but that the claims be given their broadest reasonable interpretation to cover all novel and non-obvious embodiments, literal or equivalent, disclosed or not, covered thereby.

What is claimed is:

1. A system for diagnosing a medical condition of a patient, said system comprising:
a computing device comprising first specialized software and a microphone, said computing device being configured for executing said specialized software such that the computing device collects and records sounds of the patient and the patient's surroundings while the patient is sleeping, wherein
said computing device is configured to connect to a communication network; and
an analysis device comprising second specialized software for receiving the recorded sounds collected by the computing device using the communication network, wherein said analysis device executes the second specialized software to analyze the received sounds for diagnosing a condition of sleep apnea in the patient.

2. The system of claim 1, wherein said computing device is a mobile smart phone or tablet computer of the patient and wherein said analysis device is located remotely from the patient.

3. The system of claim 1, wherein said recorded sounds of the patient include a recording of snoring sounds of the patient, and where said analysis device diagnoses the condition of sleep apnea by analyzing the snoring sounds.

4. The system of claim 3, further configured to differentiate the snoring sounds of the patient from other sounds of the patient and from background sounds.

5. The system of claim 3, wherein said analyzing of the received sounds includes determining any one or more of:
a wake period where the analysis of the received sounds indicates that there is speaking or other noises/sounds of certain minimum duration indicating that the patient is likely awake;
an apnea event where the analysis of the snoring sounds indicates that a limited duration of no snore is followed by one or a few snores, which are then followed by another limited period of no snore;
an apnea event where the analysis of the snoring sounds indicates that there is a crescendo of limited duration in a snore, followed or preceded by a no snore event of a limited duration;
an apnea event where analysis of the snoring sounds shows that there is a no snore period of limited duration;
an apnea event where the analysis of the snoring sounds shows that there is a diminuendo of limited duration in a snore, followed or preceded by a no snore period of a limited duration; and/or
an apnea event where the analysis of the snoring sounds shows that there is a crescendo or diminuendo in a snore of limited duration.

6. The system of claim 3, wherein said computing device is comprised of another sensor configured with the computing device to collect and record data other than sounds for use by the system in differentiating snoring sounds from other sounds.

7. The system of claim 1, wherein said analyzing of the received sounds includes determining any one or more of:
a wake period where the analysis of the received sounds indicates that there is speaking or other noises/sounds of certain minimum duration indicating that the patient is likely awake;
an apnea event where the analysis of the received sounds indicates that a limited duration of no snore is followed by one or a few snores, which are then followed by another limited period of no snore;
an apnea event where the analysis of the received sounds indicates that there is a crescendo of limited duration in a snore, followed or preceded by a no snore event of a limited duration;
an apnea event where analysis of the received sounds shows that there is a no snore period of limited duration;
an apnea event where the analysis of the received sounds shows that there is a diminuendo of limited duration in a snore, followed or preceded by a no snore period of a limited duration; and/or
an apnea event where the analysis of the received sounds shows that there is a crescendo or diminuendo in a snore of limited duration.

8. A system for diagnosing a medical condition of a patient, said system comprising:
a computing device comprising first specialized software and a microphone, said computing device being configured for executing said specialized software such that the computing device collects and records snoring sounds of the patient while the patient is sleeping, wherein
said system is configured for executing second specialized software to analyze the snoring sounds for diagnosing a condition of sleep apnea in the patient.

9. The system of claim 8, wherein said computing device is a mobile smart phone of the patient.

10. The system of claim 8, further configured to differentiate the snoring sounds of the patient from other sounds of the patient and from background sounds recorded by the microphone.

11. The system of claim 8, wherein said analyzing of the snoring sounds includes determining any one or more of:
a wake period where the analysis includes analyzing additional sounds that indicate that there is speaking or other noises/sounds of certain minimum duration indicating that the patient is likely awake;
an apnea event where the analysis of the snoring sounds indicates that a limited duration of no snore is followed by one or a few snores, which are then followed by another limited period of no snore;
an apnea event where the analysis of the snoring sounds indicates that there is a crescendo of limited duration in a snore, followed or preceded by a no snore event of a limited duration;
an apnea event where analysis of the snoring sounds shows that there is a no snore period of limited duration;
an apnea event where the analysis of the snoring sounds shows that there is a diminuendo of limited duration in a snore, followed or preceded by a no snore period of a limited duration; and/or
an apnea event where the analysis of the snoring sounds shows that there is a crescendo or diminuendo in a snore of limited duration.

12. The system of claim 8, wherein said computing device is comprised of another sensor configured with the computing device to collect and record additional data other than sounds for use by the system in differentiating snoring sounds from other sounds.

13. The system of claim 12, wherein said additional data includes GPS data.

14. The system of claim 12, wherein said additional data includes accelerometer data and/or gyroscope data.

15. The system of claim 12, wherein said additional data includes light data.

16. A system for diagnosing a medical condition of a patient, said system comprising:
a personal computing device comprising first specialized software, a microphone, and an additional sensor, said computing device being configured for executing said specialized software such that the personal computing device uses the microphone to collect and record snoring sounds of the patient while the patient is sleeping, and wherein said personal computing device is also configured for executing said specialized software such that the personal computing device uses the additional sensor to collect and record additional data while the patient is sleeping, and wherein said computing device is configured to connect to a communication network; and
an analysis device remotely located from said personal computing device, said analysis device comprising second specialized software for receiving the recorded snoring sounds and additional data collected by the computing device using the communication network, wherein said analysis device executes the second specialized software to analyze the snoring sounds and additional data for diagnosing a condition of sleep apnea in the patient.

17. The system of claim 16, wherein said additional data includes GPS data.

18. The system of claim 16, wherein said additional data includes accelerometer data and/or gyroscope data.

19. The system of claim 16, wherein said additional data includes light data.

20. A method of diagnosing a medical condition of a patient, said system comprising:
collecting and recording snoring sounds of a sleeping patient;
analyzing the recorded snoring sounds of the sleeping patient to detect patterns in the snoring sounds; and
diagnosing a condition of sleep apnea in the patient by analyzing the patterns in the snoring sounds.

21. The method of claim 20, wherein additional sounds are collected and recorded from which the snoring sounds are extracted.
